(19) United States
(12) Patent Application Publication
(10) Pub. No.: US 2026/0338754 A1
(43) Pub. Date: Oct. 29, 2026

(54) METHODS AND SYSTEMS FOR EMERGENCY SUPERVISION OF OVERFLOW IN SMART-CITY PIPE NETWORKS BASED ON INTERNET OF THINGS LARGE MODEL

(71) Applicant: CHENGDU QINCHUAN IOT TECHNOLOGY CO., LTD., Chengdu (CN)

(72) Inventors: Hanshu SHAO, Chengdu (CN); Junyan ZHOU, Chengdu (CN); Rui RU, Chengdu (CN)

(73) Assignee: CHENGDU QINCHUAN IOT TECHNOLOGY CO., LTD., Chengdu (CN)

(21) Appl. No.: 19/669,939

(22) Filed: May 6, 2026

(30) Foreign Application Priority Data
Apr. 7, 2026 (CN) 202610442597.8

(51) Int. Cl. G01N 33/18; G01W 1/10; G06F 30/27

(52) U.S. Cl. CPC G01N 33/18; G01W 1/10; G06F 30/27

(57) ABSTRACT

Provided is a system for emergency supervision of overflow in smart city pipe networks based on an Internet of Things (IoT) large model, the system comprising an emergency supervision and management platform, wherein the emergency supervision and management platform is configured to: obtain pipe network monitoring data via an emergency supervision object platform based on sensors deployed in a drainage pipe network; generate overflow estimation data via a prediction model based on the pipe network monitoring data and future weather data; in response to determining that the overflow estimation data satisfies an overflow condition, generate overflow control parameters based on the overflow estimation data, wherein the overflow control parameters include at least one of interception well parameters, pump station parameters, and regulation-and-storage facility parameters; and send the overflow control parameters to the emergency supervision object platform and control operation parameters of an emergency overflow device.

### CROSS-REFERENCE TO RELATED APPLICATIONS

This application claims priority to the Chinese Patent Application No. 202610442597.8, filed on Apr. 7, 2026, the contents of which are hereby incorporated by reference.

### TECHNICAL FIELD

The present disclosure generally relates to a field of gas pipeline supervision, and in particular to a method for emergency supervision of overflow in smart-city pipe networks and a system for emergency supervision of overflow in smart city pipe networks based on an Internet of Things (IoT) large model.

### BACKGROUND

In some city areas, a drainage system adopts a combined sewer system, i.e., domestic sewage and rainwater flow into the same pipeline. On sunny days, the system may normally transport sewage to a sewage treatment plant for treatment. However, during heavy rain, a large amount of rainwater flows into the pipe network, causing a total water volume to far exceed a design capacity of existing pipelines and the sewage treatment plant. To prevent city waterlogging and sewage backflow, a combined sewer drainage system usually sets overflow outlets at positions such as an interception well, allowing rainwater and sewage mixed water exceeding a treatment capacity to be directly discharged into nearby receiving water bodies such as rivers and lakes. This phenomenon is referred to as combined sewer overflow (CSO).

A CSO event may cause serious environmental problems. The overflowed rainwater and sewage mixed water is untreated and contains a large amount of suspended solids, organic pollutants, heavy metals, and pathogenic microorganisms, causing serious pollution and threats to water quality of the receiving water bodies, aquatic ecosystems, and public health.

How to accurately predict an occurrence time and a total overflow volume of a CSO event and achieve refined and proactive control of emergency overflow devices in a drainage pipe network to reduce an overflow volume and environmental pollution risk is a key technical problem urgently needing to be solved in the field of smart city construction and water environment management.

### SUMMARY

One or more embodiments of the present disclosure provide a system for emergency supervision of overflow in smart city pipe networks based on an IoT large model. The system comprises an emergency supervision and management platform. The emergency supervision and management platform is configured to: obtain pipe network monitoring data via an emergency supervision object platform based on sensors deployed in a drainage pipe network; generate overflow estimation data via a prediction model based on the pipe network monitoring data and future weather data; in response to determining that the overflow estimation data satisfies an overflow condition, generate overflow control parameters based on the overflow estimation data, wherein the overflow control parameters include at least one of interception well parameters, pump station parameters, and regulation-and-storage facility parameters, and the pump station parameters include an operating power and a pumping direction of a target pump; send the overflow control parameters to the emergency supervision object platform and control operation parameters of an emergency overflow device, including: controlling a gate opening degree of a sluice gate based on the interception well parameters, wherein the sluice gate is deployed at an overflow outlet of the interception well; controlling the target pump to operate at the pumping direction and the operating power based on the pump station parameters; controlling opening of a target pipeline valve to discharge water pumped by the target pump to a drainage region; controlling an inlet valve opening degree and an outlet valve opening degree of a regulation and storage facility based on the regulation-and-storage facility parameters; wherein the regulation and storage facility is connected to the drainage pipe network.

One or more embodiments of the present disclosure provide a method for emergency supervision of overflow in smart-city pipe networks. The method is performed by the emergency supervision and management platform. The method comprises: obtaining pipe network monitoring data via the emergency supervision object platform based on the sensors deployed in the drainage pipe network; generating the overflow estimation data via the prediction model based on the pipe network monitoring data and the future weather data; in response to determining that the overflow estimation data satisfies the overflow condition, generating the overflow control parameters based on the overflow estimation data, wherein the overflow control parameters include at least one of the interception well parameters, the pump station parameters, and the regulation-and-storage facility parameters, and the pump station parameters include the operating power and the pumping direction of the target pump; sending the overflow control parameters to the emergency supervision object platform and controlling the operation parameters of the emergency overflow device, including: controlling the gate opening degree of the sluice gate based on the interception well parameters, wherein the sluice gate is deployed at the overflow outlet of the interception well; controlling the target pump to operate at the pumping direction and the operating power based on the pump station parameters; controlling opening of the target pipeline valve to discharge water pumped by the target pump to the drainage region; controlling the inlet valve opening degree and the outlet valve opening degree of the regulation and storage facility based on the regulation-and-storage facility parameters; wherein the regulation and storage facility is connected to the drainage pipe network.

### BRIEF DESCRIPTION OF THE DRAWINGS

The present disclosure will be further described by way of exemplary embodiments, which will be described in detail with reference to the accompanying drawings. These embodiments are not limiting. In these embodiments, the same reference numerals denote the same structures, wherein:

FIG. 1 is a schematic diagram illustrating a platform structure of a system for emergency supervision of overflow in smart city pipe networks based on an IoT large model according to some embodiments of the present disclosure;

FIG. 2 is a flowchart of an exemplary process for emergency supervision of overflow in smart city pipe networks according to some embodiments of the present disclosure;

FIG. 3 is a schematic diagram illustrating a generation of overflow estimation data according to some embodiments of the present disclosure;

FIG. 4 is a flowchart of an exemplary process for determining a prediction model type according to some embodiments of the present disclosure;

FIG. 5 is a flowchart of an exemplary process for generating overflow control parameters according to some embodiments of the present disclosure.

### DETAILED DESCRIPTION

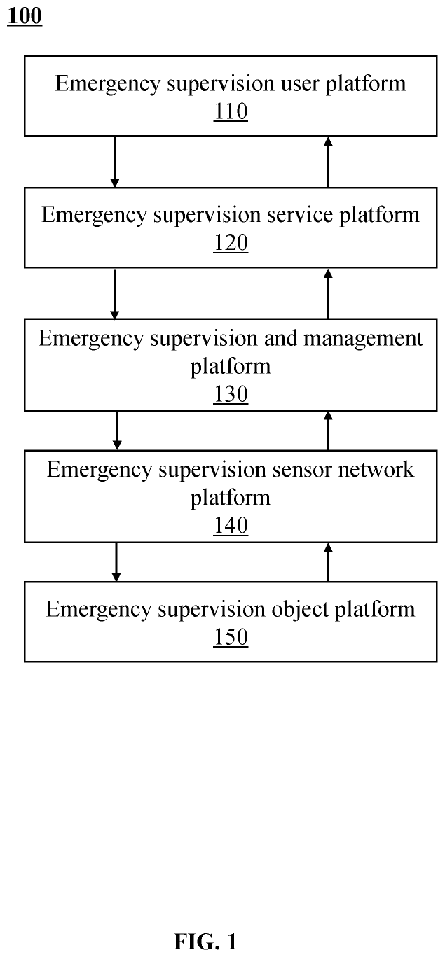

FIG. 1 is a schematic diagram illustrating a platform structure of a system for emergency supervision of overflow in smart city pipe networks based on an IoT large model according to some embodiments of the present disclosure.

In some embodiments, the system for emergency supervision of overflow in smart city pipe networks based on an IoT large model 100 may be configured to monitor, predict, warn, and perform emergency response for overflow events in an city drainage pipe network.

As shown in FIG. 1, the system for emergency supervision of overflow in smart city pipe networks based on an IoT large model 100 includes an emergency supervision user platform 110, an emergency supervision service platform 120, an emergency supervision and management platform 130, an emergency supervision sensor network platform 140, and an emergency supervision object platform 150, which are sequentially connected. In some embodiments, one or more platforms in the system for emergency supervision of overflow in smart city pipe networks based on an IoT large model 100 may exchange information and/or data via a network.

The emergency supervision user platform 110 refers to a platform configured to interact with a user. In some embodiments, the emergency supervision user platform 110 may be configured to provide data visualization and push notifications. In some embodiments, the emergency supervision user platform 110 may include various mobile terminal devices.

In some embodiments, the emergency supervision user platform 110 may perform data interaction with the emergency supervision and management platform 130 via the emergency supervision service platform 120. For example, the emergency supervision user platform 110 may obtain supervision data and information uploaded by the emergency supervision and management platform 130, or the emergency supervision user platform 110 may issue a monitoring demand instruction to the emergency supervision and management platform 130.

The emergency supervision service platform 120 refers to a platform configured to convey user instructions and control information. The emergency supervision service platform 120 may perform data interaction with the emergency supervision user platform 110 and the emergency supervision and management platform 130.

The emergency supervision and management platform 130 refers to a platform configured to supervise and manage data related to the system for emergency supervision of overflow in smart city pipe networks based on an IoT large model 100. The emergency supervision and management platform 130 may perform data interaction with the emergency supervision service platform 120 and the emergency supervision sensor network platform 140.

The emergency supervision and management platform 130 refers to a central platform responsible for overall management, data processing, decision generation, and instruction issuance for emergency supervision of overflow in city pipe networks. For example, the emergency supervision and management platform 130 may receive data from the emergency supervision object platform 150, perform overflow prediction, and generate control strategies.

In some embodiments, the emergency supervision and management platform 130 may be implemented using a plurality of hardware and software components. For example, the hardware may include a memory, computing hardware (e.g., a server, a processor, etc.), and a gateway.

In some embodiments, the emergency supervision and management platform 130 may further include a data center. The data center may include a database, a data processing model library, and a computing unit. The database is configured to store various types of data required for system operation, for example, pipe network monitoring data, future weather data, overflow estimation data, and historical control parameters. The data processing model library is configured to store various emergency supervision data processing models and prediction models. The computing unit is configured to perform data computing and model inference. The computing unit may invoke a corresponding data processing model or a prediction model from the data processing model library as needed, and retrieve corresponding data from the database for processing.

The emergency supervision sensor network platform 140 refers to a functional platform configured for sensing communication. In some embodiments, the emergency supervision sensor network platform 140 may be configured as a communication network, a gateway, etc.

In some embodiments, the emergency supervision sensor network platform 140 may interact with the emergency supervision and management platform 130 and the emergency supervision object platform 150.

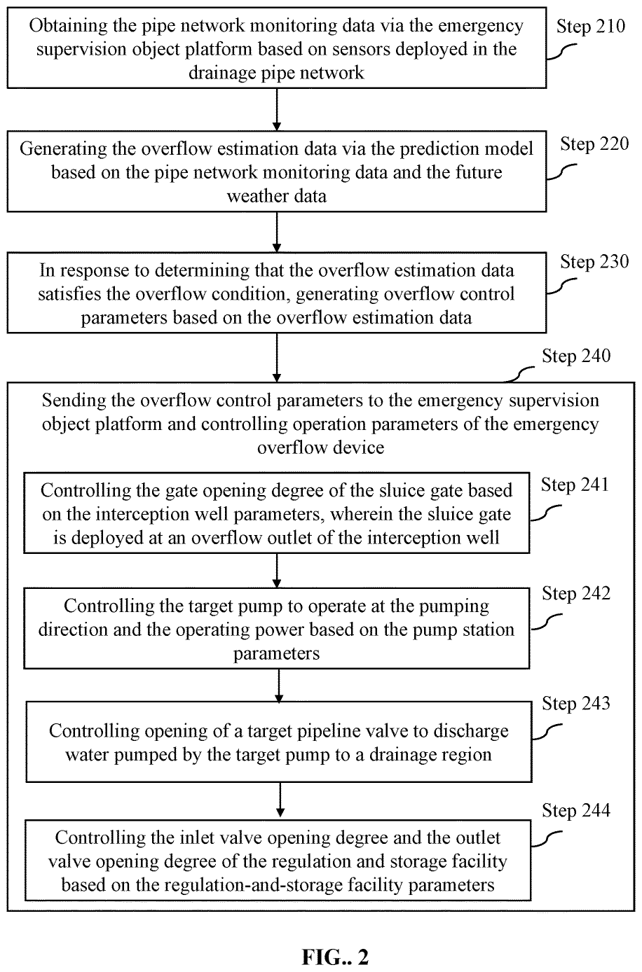

The emergency supervision object platform 150 refers to a platform responsible for collecting emergency supervision data and executing emergency control instructions. For example, the emergency supervision object platform 150 may connect to sensors deployed in the drainage pipe network to obtain the pipe network monitoring data, and control emergency overflow devices such as interception wells, pump stations, and regulation and storage facilities. More descriptions regarding the sensors may be found in the content corresponding to FIG. 2.

In some embodiments of the present disclosure, the system for emergency supervision of overflow in smart city pipe networks based on an IoT large model 100 may form an information operation closed loop among the various platforms. The system for emergency supervision of overflow in smart city pipe networks based on an IoT large model 100 may perform unified management to coordinate operations, thereby achieving informatization and intelligence for city overflow management.

FIG. 2 is a flowchart of an exemplary process for emergency supervision of overflow in smart city pipe networks according to some embodiments of the present disclosure.

Step 210, obtaining the pipe network monitoring data via the emergency supervision object platform 150 based on sensors deployed in the drainage pipe network.

The drainage pipe network refers to an underground pipeline system configured to collect, transport, and discharge city rainwater, sewage, or combined rainwater and sewage. For example, the drainage pipe network includes facilities such as pipelines, inspection wells, interception wells, and pump stations.

The sensors are configured to monitor an operating state of the drainage pipe network in real time. For example, the sensors include a rain gauge, a liquid level gauge, a flow meter, a gate opening degree sensor, a pump station status sensor, etc.

The pipe network monitoring data refers to various types of data regarding an operating state of the drainage pipe network. For example, the pipe network monitoring data includes one or more of rainfall data, water level data, flow data, or equipment operation status data. The equipment operation status data may include operation status data such as on/off state, current, power, and operating duration of equipment such as pumps.

In some embodiments, the emergency supervision and management platform obtains the pipe network monitoring data from a plurality of sensors deployed in the drainage pipe network via the emergency supervision object platform.

For example, the emergency supervision and management platform monitors rainfall intensity and cumulative rainfall from rain gauges deployed in the drainage pipe network via the emergency supervision object platform, and arranges monitoring points reasonably according to topographic distribution to comprehensively grasp regional rainfall conditions. As another example, the emergency supervision and management platform monitors information such as current, power, and operating duration of pumps from pump station status sensors deployed in the drainage pipe network via the emergency supervision object platform.

Step 220: generating the overflow estimation data via the prediction model based on the pipe network monitoring data and the future weather data.

The future weather data refers to various types of data regarding meteorological conditions for a future period of time. For example, the future weather data includes future rainfall intensity, rainfall range, rainfall duration, and air temperature. The future weather data may be provided by meteorological radar or the like.

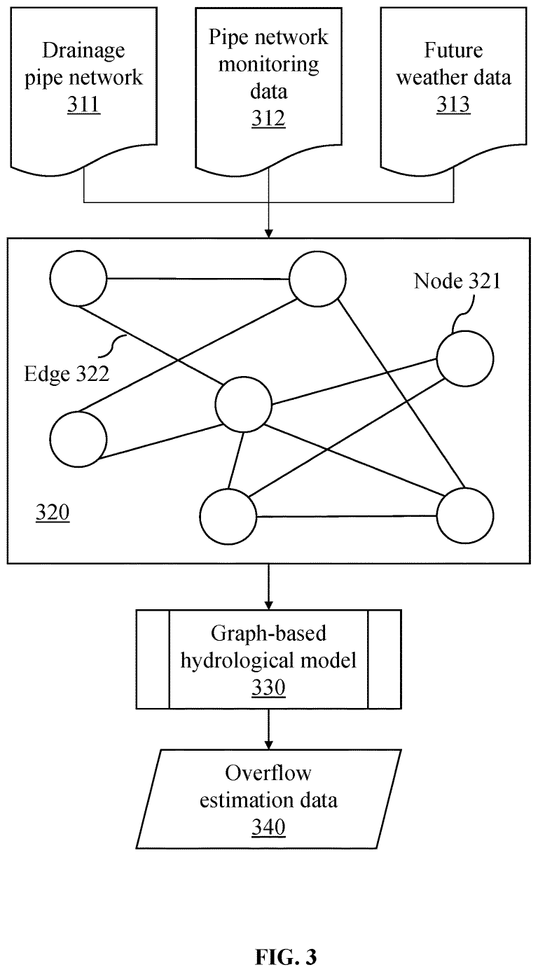

The prediction model refers to a model used for estimating or inferring future events, states, or trends based on input data. In some embodiments, the prediction model includes a hydrological simulation model for simulating hydrodynamic processes and a graph-based hydrological model for prediction based on a graph structure. More related descriptions may be found in the corresponding description in FIG. 3.

The overflow estimation data refers to data related to overflow events that are predicted to possibly occur in the future. For example, the overflow estimation data includes one or more of a predicted water level curve at a future time point, a predicted overflow occurrence location, or an overflow flow curve. The predicted water level curve refers to a curve reflecting water levels at various locations, such as drainage pipes or overflow outlets. An overflow flow curve reflects an overflow flow corresponding to each time point from overflow start to overflow end at an overflow occurrence location.

In some embodiments, the emergency supervision and management platform inputs current pipe network monitoring data as an initial condition and the future weather data as a driving condition into the prediction model, thereby driving the prediction model to perform computing to obtain the overflow estimation data. More content regarding obtaining the overflow estimation data may be found in related descriptions later in the present disclosure.

Step 230: in response to determining that the overflow estimation data satisfies the overflow condition, generating overflow control parameters based on the overflow estimation data.

The overflow condition refers to a preset condition or threshold used for determining whether an overflow event is about to occur or has already occurred. For example, the overflow condition may be that an estimated overflow volume exceeds a certain threshold, or that a pipe network water level reaches a preset warning height.

The overflow control parameters refer to control instructions or set values used for regulating emergency overflow devices to manage or reduce overflow events. The overflow control parameters include at least one of the interception well parameters, pump station parameters, or regulation-and-storage facility parameters.

The interception well parameters refer to parameters used for controlling a sluice gate or other components within the interception well to regulate water flow. For example, the interception well parameters include the gate opening degree of a sluice gate of an interception well.

The pump station parameters refer to parameters used for controlling an operating state and a water flow direction of pumps within a pump station. For example, the pump station parameters include an operating power, a start/stop state, and a pumping direction of a target pump.

The target pump refers to a specific pump selected within a pump station for performing a water pumping or drainage task.

The operating power refers to an energy consumption or output rate of equipment during operation. For example, the operating power may be electrical power consumed by a pump when pumping water flow, or may be a percentage of a rated power of the pump.

The pumping direction refers to a flow direction indication of water being pumped by a pump from one region and conveyed to another region.

The regulation-and-storage facility parameters refer to parameters used for controlling an inlet water state and an outlet water state of a regulation and storage facility. For example, the regulation-and-storage facility parameters include an inlet valve opening degree of the regulation and storage facility and an outlet valve opening degree of the regulation and storage facility.

In some embodiments, the emergency supervision and management platform determines whether the obtained the overflow estimation data satisfies a preset overflow condition. For example, when it is predicted that a future water level within the pipe network will exceed an overflow weir height of the pipe network, or when an estimated overflow volume will reach or exceed a preset warning threshold, it is determined that the overflow condition is satisfied.

In some embodiments, the emergency supervision and management platform may generate the overflow control parameters based on a preset rule. Merely by way of example, the emergency supervision and management platform may pre-divide an city drainage pipe network into a plurality of independent or semi-independent control sub-regions, and pre-set a series of “IF-THEN” control rules for the emergency overflow devices (e.g., sluice gates of interception wells, pumps of pump stations, valves of regulation and storage facilities) in each sub-region. A trigger condition of a preset rule is associated with the overflow estimation data. For example, one of the rules may be: “if a predicted water level exceeds a threshold A, set the gate opening degree of the sluice gate of the interception well to a parameter X”. As another example: “if a rising rate of a predicted water level exceeds a threshold B, start a target pump station Y”. The emergency supervision and management platform may convert a prediction result into specific control parameters by looking up a table or mapping.

In step 240, sending the overflow control parameters to the emergency supervision object platform and controlling operation parameters of the emergency overflow device.

The emergency overflow device refers to an on-site facility configured to manage water flow or reduce an impact of overflow by adjusting an operation state of the emergency overflow device when an overflow event occurs or is likely to occur. For example, the emergency overflow device includes the interception well, pump stations, regulation and storage facilities, and related sluice gates and valves.

An operation parameter refers to a specific operation setting used to define or adjust an operation of a device when the device performs a function of the device. For example, the operation parameters of the emergency overflow device include the gate opening degree of the sluice gate of the interception well, the operating power and the pumping direction of a pump in the pump station, and the inlet valve opening degree and the outlet valve opening degree of the regulation and storage facility.

In step 241, controlling the gate opening degree of the sluice gate based on the interception well parameters, wherein the sluice gate is deployed at an overflow outlet of the interception well.

The interception well refers to a structure configured to intercept and convey sewage to a sewage treatment plant when it is not raining, and allow a portion of stormwater-sewage mixed water to overflow when a water volume exceeds a carrying capacity during rainy days. For example, the interception well typically includes a pipeline connected to a sewage treatment plant and the overflow outlet at a higher elevation, and controls water flow through the sluice gate.

The gate opening degree refers to an opening degree of the sluice gate, a valve, or another regulating device, usually expressed as a percentage or an angle. For example, the gate opening degree may be 50%, indicating that the sluice gate is opened to a half position.

In some embodiments, the emergency supervision and management platform sends a control instruction to an on-site interception well through the emergency supervision object platform according to the sluice gate number and a corresponding gate opening degree value specified in the interception well parameters. After receiving the instruction, an electric actuator of a corresponding on-site sluice gate adjusts an opening degree of the sluice gate based on the gate opening degree value carried in the instruction. For example, when an upstream region is about to experience a brief but intense rainfall peak, the emergency supervision and management platform automatically reduces the gate opening degree of the sluice gate of the interception well on the premise of ensuring that a downstream sewage treatment plant is not overloaded.

In step 242, controlling the target pump to operate at the pumping direction and the operating power based on the pump station parameters.

A pump station refers to a device configured to pump water from a low place to a high place, or transfer water from one region to another region. Active intervention and scheduling of water flow within a pipe network system can be achieved by using a pump station.

In some embodiments, the emergency supervision and management platform sends a control instruction to the target pump in a pump station through the emergency supervision object platform according to a target pump number, the operating power, and the pumping direction specified in the pump station parameters, thereby controlling start and stop of the pump, adjusting an operating frequency or power of the pump, and setting the pumping direction of water flow.

In step 243, controlling opening of a target pipeline valve to discharge water pumped by the target pump to a drainage region.

The target pipeline valve refers to a specific valve used to control connection or disconnection of water flow between different pipelines or regions. For example, when a pump station pumps water from pipeline A to pipeline B, the target pipeline valve is a valve between pipeline A and pipeline B.

The drainage region refers to a designated geographical area or facility to which water is guided or discharged. For example, the drainage region may be another section of a drainage pipeline, the regulation and storage facility, or a nearby receiving water body.

In some embodiments, when the emergency supervision and management platform determines that water volume transfer needs to be performed through the target pump, the emergency supervision and management platform retrieves a database according to a pumping region and the drainage region specified in the pump station parameters, to determine the target pipeline valve that needs to be opened to connect the pumping region and the drainage region. The target pipeline valve is typically in a closed state when not pumping or draining. The emergency supervision and management platform sends an opening instruction to the valve, to form a water flow channel, ensuring that water pumped by the target pump can be smoothly discharged to a designated drainage region.

In step 244, controlling the inlet valve opening degree and the outlet valve opening degree of the regulation and storage facility based on the regulation-and-storage facility parameters.

The regulation and storage facility refers to a structure used to temporarily store rainwater or stormwater-sewage mixed water to regulate a water volume of the drainage pipe network. The regulation and storage facility is connected to the drainage pipe network. A flood peak can be reduced and overflow pollution can be reduced by using the regulation and storage facility. For example, the regulation and storage facility may include an underground storage tank or a deep tunnel, connected to the drainage pipe network through an inlet valve and an outlet valve.

The inlet valve opening degree refers to an opening degree of a valve used by the regulation and storage facility to control water entry.

An outlet valve opening degree refers to an opening degree of a valve of the regulation and storage facility for controlling discharge of water.

In some embodiments, the regulation and storage facility is configured to store rainwater far exceeding a processing capacity of a pipe network system, thereby achieving “peak shaving” and pollution reduction. The regulation and storage facility is typically connected to different pipelines of the drainage pipe network through a plurality of inlets and a plurality of outlets.

In some embodiments, the emergency supervision and management platform, based on a regulation-and-storage facility number, an inlet valve opening degree, and the outlet valve opening degree specified in the regulation-and-storage facility parameters, transmits a control instruction to an electric valve of an on-site regulation and storage facility through the emergency supervision object platform, thereby adjusting opening degrees of an inlet valve and an outlet valve to control a volume of water entering and discharging from the regulation and storage facility.

For example, when an extremely high rainfall intensity is predicted for a next hour, and severe overflow cannot be avoided regardless of adjusting sluice gates and pump stations, the regulation and storage facility may be activated. The emergency supervision and management platform determines an optimal opening timing and an optimal inlet valve opening degree, and guides initial rainwater with a highest pollution concentration (a “first wave of dirty water” that flushes ground surfaces and pipelines when rain just starts) into a pool.

In some embodiments of the present disclosure, the system for emergency supervision of overflow in smart city pipe networks based on an IoT large model, by integrating sensors deployed within the drainage pipe network, obtains multi-dimensional pipe network monitoring data in real time, combines the pipe network monitoring data with the future weather data, and utilizes the prediction model to accurately estimate potential overflow events; based on predicted overflow situations, the system dynamically adjusts hydraulic conditions of the pipe network, achieves reasonable scheduling and distribution of water volume, thereby reducing a frequency and an impact of overflow, effectively mitigating environmental pollution caused by overflow, and enhancing resilience and fine management level of an city drainage pipe network.

FIG. 3 is a schematic diagram illustrating a generation of overflow estimation data according to some embodiments of the present disclosure.

In some embodiments, the prediction model may include a hydrological simulation model (HSM) and a graph-based hydrological model. The HSM may be a physical model, and the graph-based hydrological model may be a neural network model.

In some embodiments, as shown in FIG. 3, the emergency supervision and management platform may determine a prediction horizon; determine a prediction model type based on the prediction horizon; in response to determining that the prediction model type is a graph-based hydrological model 330, construct an city pipe network graph 320 based on structural data of a drainage pipe network 311, pipe network monitoring data 312, and future weather data 313; generate overflow estimation data 340, based on the city pipe network graph 320, using the graph-based hydrological model 330.

The HSM refers to a model that simulates movement, distribution, and transformation of water in geographical space and time based on physical laws and hydrological cycle processes. For example, a Storm Water Management Model (SWMM) model, a Hydrological Simulation Program-FORTRAN (HSPF) model, etc.

The graph-based hydrological model 330 refers to a model that represents a hydrological system (e.g., the drainage pipe network) as a graph structure and utilizes graph analysis or graph neural network manners for data processing and prediction.

In some embodiments, the emergency supervision and management platform may determine the prediction horizon.

The prediction horizon refers to a length of a future time range for which prediction is required. For example, the prediction horizon may be a next 30 minutes, 1 hour, or 6 hours.

In some embodiments, the emergency supervision and management platform may determine the prediction horizon by analyzing the future weather data. For example, the emergency supervision and management platform may obtain rainfall intensity data for a specific future time period and determine an average regional rainfall intensity. If a determined average regional rainfall intensity is larger, indicating a more severe future rainfall situation, a shorter prediction horizon is preferred for a faster and more timely response. Conversely, if the average regional rainfall intensity is smaller, a longer prediction horizon may be adopted to obtain more comprehensive information. In some embodiments, the emergency supervision and management platform may determine the prediction horizon by querying a preset table based on rainfall intensity. The preset table is constructed based on rainfall intensity and the prediction horizon.

In some embodiments, the emergency supervision and management platform may determine an overflow prototype pattern based on the future weather data; determine a type of device to be controlled based on the overflow prototype pattern; and determine the prediction horizon based on the type of device to be controlled.

The overflow prototype pattern refers to a type of overflow event with typical characteristics formed by summarizing and classifying historically occurred overflow events. For example, the overflow prototype patterns include a “west city single-point sudden thunderstorm pattern”, a “rainband moving from south to north pattern”, etc.

In some embodiments, the emergency supervision and management platform may collect historical overflow event data, and summarize and classify the historical overflow event data to determine the overflow prototype pattern. For example, the emergency supervision and management platform may collect historical records of overflow events that have occurred over a long period, the historical records containing rainfall data at that time, response data of the drainage pipe network (i.e., the operation parameters for emergency overflow devices), etc.

Merely by way of example, the emergency supervision and management platform may utilize a clustering algorithm, such as K-Means, Density-Based Spatial Clustering of Applications with Noise (DBSCAN), or a hybrid manner combining an autoencoder with clustering to cluster the collected historical overflow event data, thereby obtaining the overflow prototype pattern with typical characteristics.

In some embodiments, a manner of determining the overflow prototype pattern may also be through an expert experience rule base, identification based on a deep learning model, or judgment based on preset thresholds and conditions.

In some embodiments, the emergency supervision and management platform may determine the type of device to be controlled based on the determined overflow prototype pattern.

The type of device to be controlled refers to a type of emergency overflow device that is pre-judged to require regulation in a specific overflow scenario. For example, the type of device to be controlled may be the sluice gate of the interception well, a pump of a pump station, or a valve of the regulation and storage facility. If an overflow event occurs in the future, the emergency supervision and management platform is configured to use a specific type of emergency overflow device for regulation.

In some embodiments, the emergency supervision and management platform may determine the type of device to be controlled based on the overflow prototype pattern corresponding to current or predicted future weather, and identify a type of the emergency overflow device most suitable for regulation. For example, the emergency supervision and management platform may analyze the future weather data using a classification algorithm (e.g., decision tree, support vector machine, K-nearest neighbors, or the like) to determine to which the identified overflow prototype pattern of the future weather scenario belongs.

Merely by way of example, once the current overflow prototype pattern is identified, the emergency supervision and management platform may extract a reference type of device to be controlled and a quantity of the reference type of device to be controlled corresponding to the overflow prototype pattern. For example, if the current scenario is determined to be a “west city single-point sudden thunderstorm pattern”, and historical statistical data indicates that this pattern typically requires controlling the sluice gates of interception wells with an average quantity of 30, the emergency supervision and management platform determines “sluice gates of interception wells” as the type of device to be controlled and records the corresponding quantity.

In some embodiments, the type of device to be controlled may also be determined based on expert-preset rules, based on risk assessment results, or by selecting from a plurality of device types using a multi-objective optimization algorithm.

In some embodiments, the emergency supervision and management platform may determine the prediction horizon based on the determined type of device to be controlled.

In some embodiments, the emergency supervision and management platform may set an appropriate future prediction time range based on the type of device to be controlled and a quantity of the device to be controlled, thereby determining the prediction horizon. For example, the emergency supervision and management platform may determine the prediction horizon by querying a preset table, the preset table including the type of device to be controlled and a corresponding control response time or a required prediction horizon.

Some embodiments of the present disclosure determine the overflow prototype pattern based on the future weather data, determine the type of device to be controlled based on the overflow prototype pattern, and determine the prediction horizon based on the type of device to be controlled, thereby making the prediction process more purposeful and targeted. This improves the efficiency and real-time capability of emergency decision-making, optimizes the utilization of computing resources, ensures the provision of sufficiently timely and focused overflow estimation data at critical moments, and further enhances the capability of the emergency supervision and management platform to respond to city waterlogging and overflow.

In some embodiments, the emergency supervision and management platform may determine the prediction model type based on the determined prediction horizon.

The prediction model type refers to a specific type or combination of prediction models that may be used to generate the overflow estimation data. For example, the prediction model type may be the HSM, the graph-based hydrological model, or a combination thereof.

In some embodiments, the emergency supervision and management platform may select the HSM or a graph-based hydrological model based on a length of the prediction horizon. For example, when the prediction horizon is less than or equal to a duration threshold, the graph-based hydrological model with higher computational efficiency may be selected for prediction; when the prediction horizon is greater than the duration threshold, the HSM may be selected. The duration threshold may be preset by the system.

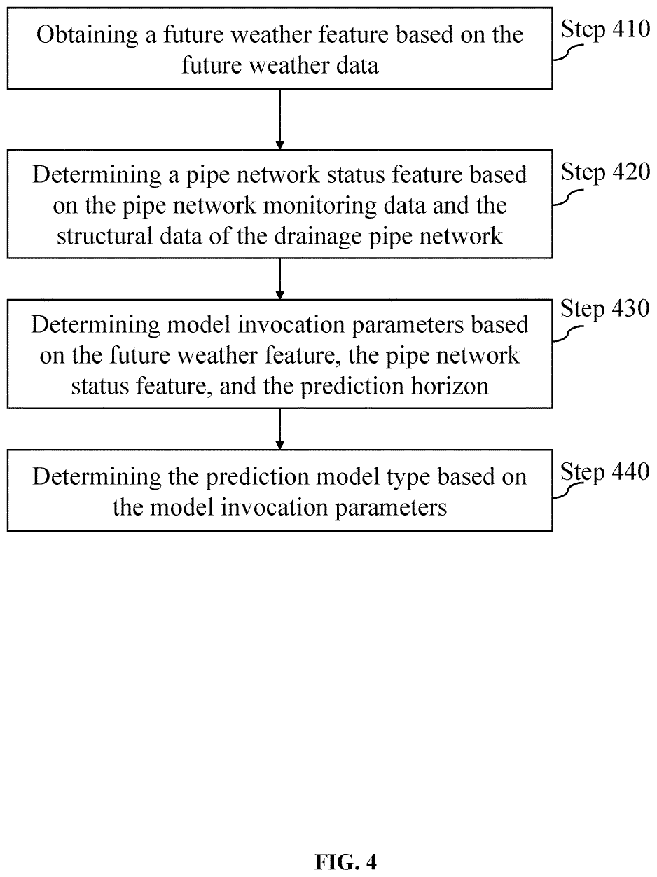

In some embodiments, the prediction model type may also be determined through a plurality of other manners. For example, in addition to considering the prediction horizon, factors such as availability of computing resources, prediction accuracy requirements, historical prediction performance, and current complexity of the pipe network may be comprehensively considered to determine the prediction model type. More descriptions regarding determining the prediction model may be found in the description corresponding to FIG. 4.

A structural data of the drainage pipe network 311 refers to various types of data describing static information of the drainage pipe network, such as physical layout, topological connections, facility dimensions, and material. For example, the structural data of the drainage pipe network includes pipe length, diameter, slope, material, and geographic locations and specifications of facilities such as inspection wells, interception wells, and pump stations.

The city pipe network graph 320 refers to graph structure data reflecting facilities (e.g., inspection wells, pump stations, the overflow outlets) and pipe connection relationships in an city drainage pipe network. The city pipe network graph 320 is composed of a plurality of nodes 321 and a plurality of edges 322.

In some embodiments, the nodes 321 may correspond to an inspection well, a pump station, the sluice gate, the valve of the regulation and storage facility, or the like, and the edges 322 may correspond to the drainage pipeline.

In some embodiments, a node feature of the node 321 includes a geographic location (e.g., latitude and longitude) of a facility corresponding to the node 321, elevation information, real-time water level data, and real-time flow data. In some embodiments, if the node 321 is a pump station, the sluice gate, or the valve of the regulation and storage facility, a node feature corresponding to the node 321 further includes a current operation parameter of the pump station, the sluice gate, or the valve of the regulation and storage facility. In some embodiments, the node feature further includes a current rainfall intensity and predicted rainfall intensities at various future time points associated with an area where the node 321 is located.

In some embodiments, an edge feature of the edge 322 includes a length, a diameter, a slope, and a material roughness of a pipeline corresponding to the edge. The material roughness may include a material type of the pipeline, such as concrete or polyvinyl chloride (PVC).

As shown in FIG. 3, the emergency supervision and management platform constructs the city pipe network graph 320 based on the structural data of the drainage pipe network 311, the pipe network monitoring data 312, and the future weather data 313.

For example, the structural data of the drainage pipe network 311 may include construction drawings, engineering drawings, geographic information system (GIS) data, or the like, of the city pipe network. The structural data describes pipe length, diameter, slope, material, and geographic locations and specifications of facilities such as inspection wells, interception wells, pump stations, the overflow outlets, and regulation and storage facilities. The emergency supervision and management platform may read the structural data from a database.

In some embodiments, the emergency supervision and management platform may traverse the structural data or the GIS data to abstract major facilities in the drainage pipe network, such as each pump station, interception well, overflow outlet, regulation and storage facility, and key inspection well, as nodes of the city pipe network graph. Each node is assigned a unique identifier (ID).

In some embodiments, the emergency supervision and management platform may establish connection relationships between the abstracted nodes based on pipe connection relationships described in the structural data of the drainage pipe network or the GIS data, forming edges of the graph. For example, if water can flow from facility A to facility B through a pipeline, a directed edge from A to B is established.

In some embodiments, the emergency supervision and management platform may determine pipe network health data based on the pipe network monitoring data; construct the city pipe network graph based on the pipe network health data, the structural data of the drainage pipe network, the pipe network monitoring data, and the future weather data.

The pipe network health data refers to various types of data for assessing structural integrity, operational efficiency, and potential failure risks of pipelines or devices in the drainage pipe network. For example, the pipe network health data includes a sediment accumulation degree of a pipeline, a damage condition of the pipeline, or a blockage risk level of the pipeline.

In some embodiments, determining the pipe network health data may include performing real-time or periodic analysis based on the pipe network monitoring data. More descriptions about the pipe network monitoring data may be found in the corresponding description above.

For example, the emergency supervision and management platform may use a preset rule base to identify an abnormal condition, thereby determining the pipe network health data. Merely by way of example, if a nighttime flow rate of a certain pipe section continuously exceeds a preset threshold, the emergency supervision and management platform may determine that pipeline damage or groundwater infiltration may exist. The emergency supervision and management platform may elevate a damage risk level of the pipe section (e.g., from a low risk to a medium risk) as part of the pipe network health data.

In some embodiments, constructing the city pipe network graph involves integrating a plurality of data sources into a unified, visual, or queryable data model.

For example, in one embodiment, the emergency supervision and management platform first uses the structural data of the drainage pipe network (e.g., a length of a pipeline, a diameter of the pipeline, a slope of the pipeline, a material of the pipeline, and a connection relationship of the pipeline) to establish a basic topological structure of the city pipe network, including nodes (e.g., inspection wells, pump stations) and edges (e.g., the drainage pipelines).

On this basis, the emergency supervision and management platform further attaches the pipe network health data, the pipe network monitoring data (e.g., real-time water levels, flow rates, rainfall amounts), and the future weather data as dynamic attributes or associated information to corresponding nodes or edges of the pipe network graph.

In some embodiments of the present disclosure, by determining the pipe network health data based on the pipe network monitoring data, and constructing the city pipe network graph together with the structural data of the drainage pipe network, the pipe network monitoring data, and the future weather data, a real-time operation status and a health status of an city drainage pipe network can be dynamically and comprehensively reflected. By dynamically updating the pipe network graph, long-term accuracy of subsequent prediction models is enhanced, and long-term reliability of prediction results is ensured.

As shown in FIG. 3, inputs to the graph-based hydrological model 330 include the city pipe network graph 320, and the graph-based hydrological model 330 outputs the overflow estimation data 340.

In some embodiments, the graph-based hydrological model includes a graph convolutional network (GCN), a graph attention network (GAN), or other graph neural network (GNN) models.

In some embodiments, the emergency supervision and management platform may obtain a training dataset to train the graph-based hydrological model. The training dataset includes training samples and corresponding labels of the training samples. A training sample may be a historical city pipe network graph sample, and a label is an actual water level curve, an overflow occurrence location, and an overflow flow rate curve of the overflow occurrence location at a subsequent time point collected through monitoring. The subsequent time point refers to a time point within the prediction horizon starting from a current time point. The water level curve includes water level changes at various locations such as drainage pipelines and the overflow outlets. The overflow flow rate curve includes a predicted overflow volume at each time point from a start of an overflow to an end of the overflow. The historical city pipe network graph sample and the label may be obtained based on historical data.

In some embodiments, a training process of the graph-based hydrological model includes performing a plurality of iterations. At least one iteration of the plurality of iterations includes: selecting one or more training samples from the training dataset; inputting the one or more training samples into an initial graph-based hydrological model to obtain a predicted output corresponding to the one or more training samples; substituting the predicted output corresponding to the one or more training samples and the label(s) of the one or more training samples into a formula of a predefined loss function to determine a value of the loss function; and updating model parameters in the initial graph-based hydrological model in reverse based on the value of the loss function through a manner such as gradient descent. When an iteration end condition is satisfied, an iteration process ends, and a trained model is obtained.

In some embodiments of the present disclosure, by introducing the graph-based hydrological model as the prediction model type and dynamically selecting the prediction model according to the prediction horizon, efficiency and adaptability of overflow prediction for city drainage pipe networks are significantly improved. A strategy of intelligently selecting a model according to prediction requirements optimizes utilization of computing resources, provides reliable and timely overflow estimation data, thereby providing more flexible and accurate decision support for the emergency supervision and management platform, and reducing risks caused by city waterlogging and overflow.

In some embodiments, the emergency supervision and management platform may, in response to determining that the pipe network health data indicates that the drainage pipeline satisfies a sediment cleaning condition, determine a flushing time based on the future weather data. At the flushing time, the emergency supervision and management platform may generate a flushing control instruction and send the flushing control instruction to a flushing valve associated with the drainage pipeline to perform a pipeline flushing operation.

The drainage pipeline refers to a specific pipeline segment in the drainage pipe network used for conveying water. For example, the drainage pipeline may be a main pipe for collecting domestic sewage or a branch pipe for conveying rainwater.

The sediment cleaning condition refers to a preset standard or threshold for determining whether sediment, debris, or the like is accumulated inside the drainage pipeline and whether sediment cleaning maintenance is required. For example, the sediment cleaning condition may be an abnormal rise in a water level inside the pipeline at a low flow rate, or the pipe network health data indicating that a blockage degree exceeds a certain risk level. As another example, the sediment cleaning condition may be that a blockage degree of the pipe network is greater than a preset threshold.

The flushing time refers to a preset time point or time period suitable for performing the pipeline flushing operation on the drainage pipeline. For example, the flushing time may be a nighttime low-flow period or a period predicted to have no rainfall.

In some embodiments, when the pipe network health data indicates that the drainage pipeline satisfies the sediment cleaning condition, the emergency supervision and management platform determines an appropriate flushing time based on the future weather data. The sediment cleaning condition may be that, in the pipe network health data, a blockage degree score of the drainage pipeline exceeds a preset threshold.

For example, when the pipe network health data indicates that the drainage pipeline satisfies the sediment cleaning condition, the emergency supervision and management platform uses a period predicted to have no rainfall in the future weather data as a preferred flushing time.

The flushing control instruction refers to a instruction sent to the flushing valve or other related devices for initiating, adjusting, or stopping the pipeline flushing operation. For example, the flushing control instruction may include an opening time of the flushing valve, a flushing pressure, and a flushing duration.

The flushing valve refers to a dedicated valve deployed on the drainage pipeline for controlling a water source to flush the pipeline and remove internal sediment. For example, the flushing valve may be connected to an offline flushing well or use a water source from an upstream main pipe network for pipeline flushing.

The pipeline flushing operation refers to a maintenance activity of injecting a high-flow water body into the drainage pipeline to clear internal sediment, sand, and debris. For example, the pipeline flushing operation is typically performed during non-peak flow periods to restore a water conveyance capacity of the pipeline.

In some embodiments, the emergency supervision and management platform generates a corresponding flushing control instruction at the flushing time. The flushing control instruction typically includes parameters for operating the flushing valve, such as a valve opening time, a flushing duration, and a flushing pressure.

For example, the emergency supervision and management platform dynamically adjusts flushing parameters in the flushing control instruction according to a blockage degree of the drainage pipeline. If the pipe network health data shows that the pipeline is severely silted, the flushing control instruction may set a longer flushing duration and/or a higher flushing pressure to ensure a sediment cleaning effect.

The flushing valve associated with the drainage pipeline includes a special valve installed at a specific location of the pipe network (e.g., upstream of a gentle pipe section prone to siltation).

In another embodiment, the emergency supervision and management platform sends the flushing control instruction to the flushing valve associated with a designated drainage pipeline. After receiving the flushing control instruction, the flushing valve performs the pipeline flushing operation according to parameters in the flushing control instruction. For example, the valve automatically opens to allow the high-flow water body to enter the drainage pipeline, thereby flushing and clearing internal sediment and debris of the pipeline and restoring the water conveyance capacity of the pipeline. The flushing operation is typically performed during non-peak flow periods to minimize impact on daily drainage system operations.

In some embodiments, in addition to directly controlling valve opening, the flushing control instruction may also be used to adjust a flow rate or an on-off frequency of the flushing valve to adapt to different pipeline conditions and environmental requirements.

In some embodiments of the present disclosure, intelligently determining the flushing time in combination with the future weather data, and generating and sending the flushing control instruction to perform the pipeline flushing operation, not only is a timely and precise response to sediment accumulation problems in the drainage pipeline achieved, but also optimizes an implementation timing of the flushing operation by considering weather and flow factors, reducing impact on daily drainage system operations. Efficiency and safety of sediment cleaning maintenance are improved, thereby significantly enhancing overall emergency management capability and resilience of the city drainage pipe network.

FIG. 4 is a flowchart of an exemplary process for determining a prediction model type according to some embodiments of the present disclosure.

Step 410, obtaining a future weather feature based on the future weather data.

The future weather feature refers to a quantitative attribute or indicator extracted from the future weather data for describing future meteorological conditions. For example, the future weather feature includes rainfall coverage breadth, flood peak intensity, rainfall center mobility, and rainfall volatility.

In some embodiments, the emergency supervision and management platform may divide a service area of an city drainage pipe network into a plurality of grids, and determine and obtain the future weather feature according to a rainfall intensity value of each grid at each future time step or time point.

In some embodiments, the emergency supervision and management platform may also obtain the future weather feature in other ways. For example, the emergency supervision and management platform may obtain more future weather features by analyzing other meteorological elements such as temperature, wind speed, and humidity in the future weather data, or by combining GIS data.

Step 420, determining a pipe network status feature based on the pipe network monitoring data and the structural data of the drainage pipe network.

The pipe network status feature refers to a quantitative attribute or indicator for describing a current operating condition of the drainage pipe network. For example, the pipe network status feature includes network connectivity, pipe network saturation, hydraulic gradient consistency, and control device activity.

In some embodiments, the emergency supervision and management platform may extract information such as water level, flow rate, and pump station operating status from real-time or historical pipe network monitoring data, and determine the information in combination with structural data such as a topological structure, a pipe size, and a gradient of the drainage pipe network to determine the pipe network status features.

For example, the emergency supervision and management platform may determine the network connectivity by calculating an average node degree of an city pipe network graph (reflecting a connection tightness between parts of the drainage pipe network). The emergency supervision and management platform may determine the pipe network saturation by calculating a proportion of a count of monitoring nodes where a water level exceeds a warning line (e.g., exceeding 80% of a pipe diameter). The emergency supervision and management platform may determine the hydraulic gradient consistency by calculating a proportion of a count of pipelines where an actual hydraulic gradient direction is opposite to a physical gradient direction of the pipeline. The emergency supervision and management platform may determine the control device activity by calculating a total number of pump stations in an “on” or “working” state and partially closed sluice gates and valves (i.e., regulation-and-storage facilities).

In some embodiments, the emergency supervision and management platform may also determine the pipe network status features in other ways. For example, more dimensional pipe network status features may be determined according to water quality data, foreign object accumulation, and pipeline aging degree in the pipe network.

Step 430, determining model invocation parameters based on the future weather feature, the pipe network status feature, and the prediction horizon.

The model invocation parameter refers to a parameter set for guiding a selection and an execution scheme of the prediction model. For example, the model invocation parameter includes a specific invocation time and the prediction model type (also referred to as a type of the prediction model(s)) corresponding to the specific invocation time.

The invocation time refers to a specific time point or time period at which a designated prediction model is activated or a prediction result of the designated prediction model is used during a prediction process. For example, an invocation time corresponding to the HSM is a future 0 -10 minutes.

In some embodiments, the emergency supervision and management platform may generate the model invocation parameters through a prediction selection model. The prediction selection model may be a machine learning model, e.g., a multilayer perceptron (MLP), a gradient boosting decision tree (XGBoost), or Light Gradient Boosting Machine (LightGBM).

Inputs of the prediction selection model include the future weather feature (e.g., the rainfall coverage breadth, the flood peak intensity, etc.), the pipe network status features (e.g., the network connectivity, the pipe network saturation, etc.), and the prediction horizon.

An output of the prediction selection model includes the model invocation parameters, i.e., an invocation scheme for the prediction model. For example, the output may be an invocation of a graph neural network (GNN) model alone, an invocation of the HSM alone, or a combined invocation scheme.

Training samples of the prediction selection model include sample pipe network status features, sample future weather feature, and a sample prediction horizon. A label of the prediction selection model is a corresponding model invocation parameter.

The training samples and the label of the prediction selection model may be obtained based on historical data. For example, for historical events where overflow was successfully suppressed or overflow effects were satisfactory in historical data, corresponding sample pipe network status features, sample future weather feature, and sample prediction horizon (which may be an actual historical prediction horizon) are extracted. The GNN and the HSM are used to back-test the historical events, respectively, to obtain, for each historical event, errors between prediction results of the models and actually recorded data at each time point under different prediction horizons. Two error curves are plotted, and an intersection point of the error curves is found. A time corresponding to the intersection point is an optimal switching point for the historical event. The label may be using a model with a smaller error before the time of the optimal switching point, or using another model with a smaller error after the time of the optimal switching point.

A training process of the prediction selection model may be similar to a training process of the graph-based hydrological model.

In some embodiments, the determination of the model invocation parameters may also be implemented through preset rules, an expert system, reinforcement learning, or the like. For example, an invocation of a specific model may be directly triggered according to a preset rainfall intensity level and a pipe network saturation threshold.

In some embodiments, the emergency supervision and management platform may, in response to determining that a count of determined prediction model types is greater than one, combine the overflow estimation data predicted by the determined prediction models according to invocation times to obtain updated overflow estimation data.

In some embodiments, the emergency supervision and management platform may determine a count of types of prediction models currently available or activated according to a preset configuration or a real-time system status. For example, a plurality of prediction models such as the graph-based hydrological model, the HSM, and a deep learning-based graph neural network model may be configured. When at least two of the plurality of prediction models are activated or selected for prediction, the count of prediction model types is considered to be greater than one.

In some other embodiments, the emergency supervision and management platform may obtain a list of prediction models currently running or about to run by querying a processor or data in a database. If the list includes prediction models belonging to different types and a count of the prediction models exceeds one, a determination may be made that the count of prediction model types is greater than one. For example, when the GNN model and the HSM model are used for prediction simultaneously, the count of model types is two.

In some embodiments, the system may also dynamically determine whether to enable a plurality of prediction models of different types to improve prediction accuracy based on historical data or expert experience.

In some embodiments, in response to determining that a count of determined prediction model types is greater than one, the emergency supervision and management platform combines the overflow estimation data predicted by the determined prediction models according to invocation times to obtain the updated overflow estimation data.

In some embodiments, the emergency supervision and management platform receives the overflow estimation data output by different prediction models. The overflow estimation data may be for different time periods or different prediction targets. The emergency supervision and management platform integrates the overflow estimation data according to an invocation timestamp of each model and a preset combination strategy. For example, if the GNN model is for short-term prediction and the HSM model is for medium-term and long-term prediction, the emergency supervision and management platform splices a short-term prediction result of the GNN model with a medium-term and long-term prediction result of the HSM model according to a time axis.

In some embodiments, the emergency supervision and management platform may combine a prediction result of the graph-based hydrological model in the short term with a prediction result of the HSM in the medium and long-term.

In some embodiments, at a “seam” between prediction results of different models, the emergency supervision and management platform may apply a smoothing algorithm to avoid abrupt jumps in a prediction curve.

The emergency supervision and management platform may also combine the overflow estimation data predicted by the determined prediction models in various other ways, including but not limited to linear combination, nonlinear combination, decision tree combination, etc.

In some embodiments, after performing the combination operation described above, the emergency supervision and management platform may generate and obtain the updated overflow estimation data. The data are prediction results that integrate advantages of a plurality of models. The data includes overflow prediction information over a longer time horizon or with higher accuracy.

For example, after completing combination and smoothing processing of the graph-based hydrological model (GNN model) and the physical model (HSM model), the emergency supervision and management platform internally stores and provides a complete, smooth, cross-time-period predicted water level curve or overflow flow curve, which is the updated overflow estimation data.

In some embodiments of the present disclosure, when a quantity of applied prediction model types is greater than one, prediction results of different prediction models (e.g., the GNN model and the HSM model) in respective advantageous time periods can be dynamically integrated. The dynamic integration not only improves quality and naturalness of overflow prediction results but also enhances stability of a downstream optimization algorithm, providing more reliable decision support for the emergency supervision and management platform.

Step 440, determining the prediction model type based on the model invocation parameters.

In some embodiments, the emergency supervision and management platform determines the prediction model type corresponding to the model invocation parameters as the prediction model type.

More descriptions related to the prediction model type may be found in content corresponding to FIG. 2.

In some embodiments of the present disclosure, by dynamically determining the model invocation parameters based on the future weather feature, the pipe network status feature, and the prediction horizon, the emergency supervision and management platform can intelligently select or combine prediction models according to specific scenarios. The intelligent selection or combination significantly improves the accuracy and timeliness of overflow prediction for the drainage pipe network, providing more precise and timely decision support for emergency management.

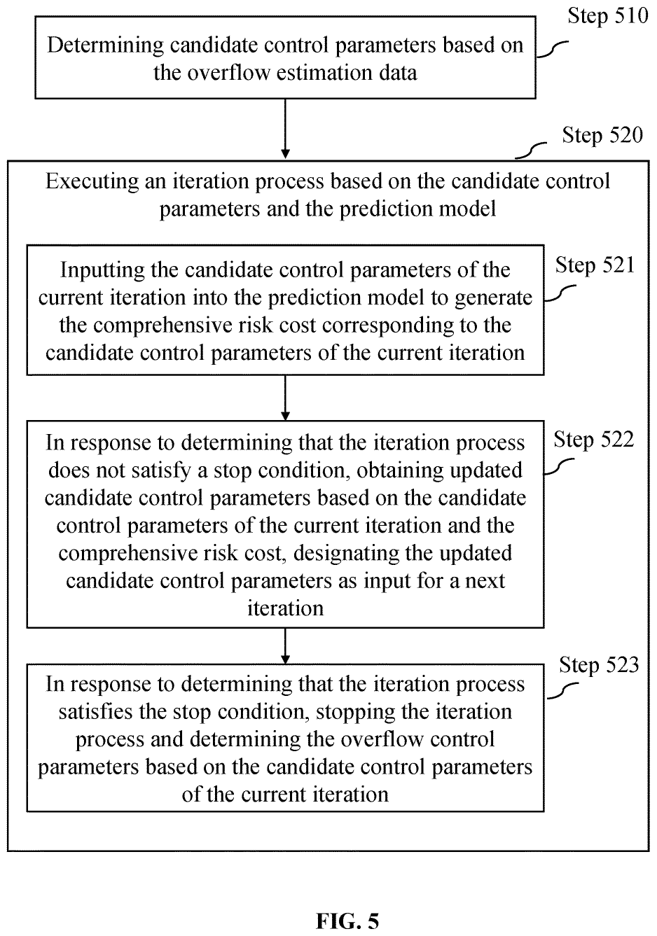

FIG. 5 is a flowchart of an exemplary process for generating overflow control parameters according to some embodiments of the present disclosure.

Step 510, determining candidate control parameters based on the overflow estimation data.

More descriptions related to the overflow estimation data may be found in descriptions corresponding to FIG. 2.

The candidate control parameters refer to parameter configurations that are evaluated during an optimization process and may be used to adjust the operation parameters of the emergency overflow device. For example, the candidate control parameters may be a combination of the gate opening degree of the interception well, the operating power of the pump station, and the valve opening degree of the regulation and storage facility randomly generated at a beginning of iterative optimization.

In some embodiments, the emergency supervision and management platform generates the candidate control parameters according to preset rules or based on historical data analysis. For example, one or more initial equipment control parameter combinations may be generated as the candidate control parameters based on equipment operation strategies under historical rainfall events or based on preliminary rules of thumb.

Step 520, executing an iteration process based on the candidate control parameters and the prediction model.

The iteration process refers to an optimization manner that repeatedly executes specific computing steps. An objective of the iteration process is to minimize a comprehensive risk cost. Each step of the iteration process improves based on a result of a previous step until a preset stopping condition is reached. For example, in a particle swarm optimization (PSO) algorithm, the iteration process includes evaluating a fitness of each of the candidate control parameters, updating individual and global best positions, and adjusting the candidate control parameters accordingly.

In some embodiments, the iteration process may be implemented using the PSO algorithm or other similar swarm intelligence optimization algorithms, e.g., a genetic algorithm (GA), a simulated annealing (SA) algorithm, etc.

The iteration process includes steps 521 to 523 below.

Step 521, inputting the candidate control parameters of the current iteration into the prediction model to generate the comprehensive risk cost corresponding to the candidate control parameters of the current iteration.

The candidate control parameters of the current iteration refer to a specific set of the candidate control parameters that are input into the prediction model for evaluation and computing at a current stage of the iteration process. For example, in an Nth iteration of the PSO algorithm, the candidate control parameters of the current iteration are a set of equipment parameter configurations represented by a current particle swarm.

In some embodiments, the emergency supervision and management platform may input the candidate control parameters of the current iteration in each round into the prediction model. The prediction model outputs the overflow estimation data corresponding to the candidate control parameters of the current iteration in the round. The predicted overflow volume of each round may be determined based on the overflow estimation data output in the round. In some embodiments, the comprehensive risk cost is determined as a fitness for evaluating the candidate control parameters.

The comprehensive risk cost refers to an indicator that quantitatively evaluates comprehensive impacts in multiple aspects such as environment, operation, and economy, brought by a specific overflow control scheme. A smaller comprehensive risk cost indicates a better effect of a corresponding overflow control scheme.

In some embodiments, the emergency supervision and management platform may determine the comprehensive risk cost by weighted calculation based on factors including the predicted overflow volume, an environmental risk weight, an equipment operating cost, and a pollutant concentration.

In some embodiments, the emergency supervision and management platform determines the comprehensive risk cost based on the predicted overflow volume of the overflow outlet output by the prediction model, the environmental risk weight, and the equipment operating cost.

The predicted overflow volume refers to an estimation of a total volume of water discharged from the overflow outlet. For example, the predicted overflow volume may include a volume of stormwater-sewage mixed water.

The environmental risk weight refers to a relative risk coefficient assigned to an overflow event occurring at the overflow outlet based on factors including importance, environmental sensitivity, and environmental capacity of a receiving water body into which the overflow outlet discharges. For example, the overflow outlet discharging into a drinking water source protection area may have a higher environmental risk weight, while the overflow outlet discharging into an ordinary river may have a lower environmental risk weight. The environmental risk weight may be preset based on experience.

The equipment operating cost refers to energy consumption, maintenance, or other related expenses incurred by the emergency overflow device during execution of a control operation.

In some embodiments, the equipment operating cost may also include equipment wear, maintenance costs, and manual operation costs. The equipment operating cost may be determined based on the operating power of the emergency overflow device, an operating time, and a switching frequency of sluice gates and valves.

In some embodiments, the comprehensive risk cost may also be determined through a multi-objective optimization function. The multi-objective optimization function includes multiple sub-objectives such as minimizing the predicted overflow volume, minimizing the equipment operating cost, and minimizing the environmental risk weight.

In some embodiments, the comprehensive risk cost may be positively correlated with an predicted overflow volume of each overflow outlet, a corresponding environmental risk weight, and an operating cost of each emergency overflow device. For example, the comprehensive risk cost may be determined by the following formula (1).

C=w₁×Σ(Vᵢ×ERWᵢ)+w₂×ΣC_{equipment} (1)

In the above formula (1), C denotes the comprehensive risk cost, w₁ denotes an adjustable weight coefficient corresponding to the predicted overflow volume, w₂ denotes an adjustable weight coefficient corresponding to the equipment operating cost, Vᵢ denotes an predicted overflow volume of an i-th overflow outlet, ERWi denotes an environmental risk weight of the i-th overflow outlet, and C_{equipment} denotes the equipment operating cost. If more emphasis is placed on minimizing a total predicted overflow volume, w₁ may be set to a relatively larger value.

In some embodiments, the emergency supervision and management platform may evaluate the environmental risk weight according to predefined rules. For example, if overflow from the overflow outlet flows into a river, an environmental risk weight of the overflow outlet may be less than an environmental risk weight of the overflow outlet whose overflow flows into a lake.

Step 522, in response to determining that the iteration process does not satisfy a stop condition, obtaining updated candidate control parameters based on the candidate control parameters of the current iteration and the comprehensive risk cost, designating the updated candidate control parameters as input for a next iteration.

The stopping condition refers to a preset standard or rule used to terminate execution of the iteration process or the optimization algorithm. For example, the stopping condition may be reaching a maximum iteration count, the comprehensive risk cost converging to a certain degree, or the comprehensive risk cost being less than a preset threshold.

In some embodiments, in the iteration process, the emergency supervision and management platform first determines whether the iteration process satisfies the stopping condition. If the iteration process does not satisfy the stopping condition, the iteration continues to search for a better control scheme. Obtaining updated candidate control parameters refers to adjusting a position and a velocity of each particle in a particle swarm based on a result of a current iteration to generate candidate control schemes for a next iteration.

Step 523, in response to determining that the iteration process satisfies the stop condition, stopping the iteration and determining the overflow control parameters based on the candidate control parameters of the current iteration.

More descriptions of the overflow control parameters may be found in the description corresponding to FIG. 2.

In some embodiments, the emergency supervision and management platform may continuously perform iterations until the iteration process satisfies the preset stop condition.

In some embodiments, the emergency supervision and management platform determines an optimal solution obtained by an iterative optimization algorithm as the overflow control parameters. In a particle swarm optimization algorithm, a global best position Gbest of the particle swarm is output as final overflow control parameters.

In some embodiments, the sensor further includes a water quality sensor. The water quality sensor is configured to obtain water quality sensor data. In some embodiments, the emergency supervision and management platform determines the pollutant concentration based on the water quality sensor data and the predicted overflow volume through a water quality prediction algorithm. The emergency supervision and management platform determines the comprehensive risk cost based on the pollutant concentration.

The water quality sensor refers to a device for detecting various chemical, physical, or biological indicators in a water body. For example, the water quality sensor may be used to measure a chemical oxygen demand (COD) concentration, a total suspended solids (TSS) concentration, or an ammonia nitrogen concentration in the water body.

The water quality sensor data refers to various data about water quality parameters obtained in real time through the water quality sensor. For example, the water quality sensor data includes a COD concentration, a TSS concentration, and an ammonia nitrogen concentration in the water body.

In some embodiments, the water quality sensor may be deployed at key nodes of a pipe network system, for example, inside the interception well at the upstream of the overflow outlet, at an inlet end of a pump station, or at an outlet end of the regulation and storage facility, to monitor water quality conditions in real time.

The water quality prediction algorithm refers to a computing manner for estimating or inferring the pollutant concentration or other water quality indicators in a water body based on input data. For example, the water quality prediction algorithm may predict the pollutant concentration by using historical water quality sensor data, the pipe network health data, and the predicted overflow volume through a machine learning model or vector database matching.

The pollutant concentration refers to a content level of a specific harmful substance in a water body. For example, the pollutant concentration may be a content of COD, TSS, or ammonia nitrogen in water at the overflow outlet.

The emergency supervision and management platform may construct a pollution feature vector based on real-time or near-real-time water quality sensor data of the overflow outlet, a total predicted overflow volume at a well outlet output by the prediction model, and an average rainfall intensity of a current rainfall event.

In some embodiments, a plurality of reference pollution feature vectors and a plurality of corresponding reference pollutant concentrations are pre-stored in a vector database. The reference pollutant concentrations are obtained based on historical overflow events.

In some embodiments, the emergency supervision and management platform first filters out all CSO events that have occurred and have complete monitoring data from a data center of the system. The emergency supervision and management platform constructs corresponding reference pollution feature vectors based on the filtered CSO overflow events. A reference pollution feature vector includes the water quality sensor data, an overflow volume, and rainfall intensity at that time. The emergency supervision and management platform uses pollutant concentrations actually monitored during these CSO overflow events (which may be an average value or a weighted average value, where a higher hazard corresponds to a higher weight) as the reference pollutant concentrations corresponding to the reference pollution feature vectors. Finally, the emergency supervision and management platform places the plurality of reference pollution feature vectors and the corresponding reference pollutant concentrations into a vector database (e.g., Milvus, Faiss, etc.) to complete construction of the vector database.

When the pollutant concentration of a current overflow outlet needs to be determined, a similarity between a current pollution feature vector and each reference pollution feature vector in the vector database is determined to find one or more most similar reference vectors. The pollutant concentration of the current overflow outlet is inferred based on the reference pollutant concentrations corresponding to the one or more most similar reference vectors.

In some embodiments, the pollutant concentration may be an average value or a weighted average value of concentrations of these harmful substances. For example, a higher weight may be assigned to a pollutant with stronger toxicity or greater environmental hazard. In some embodiments, the pollutant concentration may also be dynamically corrected based on real-time environmental monitoring data, upstream pollution source discharge information, etc.

In some embodiments, the emergency supervision and management platform may also determine the pollutant concentration based on the water quality sensor data and the pipe network health data through the water quality prediction algorithm.

That is, the pollution feature vector includes the pipe network health data of a pipeline corresponding to the overflow outlet. The corresponding pipeline may be a pipeline directly connected to the overflow outlet.

More descriptions of the water quality sensor data, the pipe network health data, the water quality prediction algorithm, the pollution feature vector, and the pollutant concentration may be found in the corresponding descriptions above.

In some embodiments of the present disclosure, by introducing health data of the pipeline corresponding to the overflow outlet into the pollution feature vector, systematic deviation in scenarios such as heavy rainfall, sudden scouring of sediment accumulation, or groundwater level fluctuations is reduced. The method reduces scheduling errors and environmental risks caused by water quality estimation errors.

In some embodiments, to more comprehensively evaluate the comprehensive impact caused by overflow, the pollutant concentration factor is further introduced on a basis of the comprehensive risk cost determined based on the predicted overflow volume of the overflow outlet output by the prediction model, the environmental risk weight (ERW), and the equipment operating cost.

In some embodiments, the comprehensive risk cost may be positively correlated to an overflow volume of each overflow outlet, the corresponding environmental risk weight, an operating cost of each device, and a product of the overflow volume of each overflow outlet and a corresponding pollutant concentration. For example, the comprehensive risk cost may be determined by the following equation (2).

C=w₁×Σ(Vᵢ×ERWᵢ)+w₂×ΣC_{equipment}+w₃×Σ(Vᵢ×Cᵢ) (2)

In the above equation (2), w₃ denotes an adjustable weight coefficient corresponding to the pollutant concentration. If pollution control is more important, a value of w₃ may be set larger. Cᵢ denotes an estimated pollutant concentration at an i-th overflow outlet during an overflow period, which may be a single-item concentration of harmful substances such as COD, TSS, ammonia nitrogen, or a weighted average value thereof. Descriptions of other coefficients may be found in the description corresponding to the above equation (1).

Some embodiments of the present disclosure obtain the water quality sensor data by introducing the water quality sensor, determine the pollutant concentration of the overflow outlet by combining the predicted overflow volume through the water quality prediction algorithm, and incorporate the pollutant concentration into the determination of the comprehensive risk cost. The method significantly improves environmental pertinence and economic effectiveness of an overflow control strategy, enabling the emergency supervision and management platform to make more intelligent and environmentally responsible decisions.

Some embodiments of the present disclosure combine the overflow estimation data output by the prediction model with the environmental risk weight and the equipment operating cost to construct the comprehensive risk cost function. The method can comprehensively and quantitatively evaluate advantages and disadvantages of different overflow control schemes, effectively improving efficiency, robustness, and control effect of an optimization algorithm. The method can effectively cope with complex and variable situations of an city stormwater pipe network under different rainfall scenarios. The method reduces environmental risks of overflow while also optimizing the equipment operating cost. The method helps reduce risks of city waterlogging and environmental pollution, and improves an intelligent level of emergency response.

The basic concepts have been described above. Obviously, for a person skilled in the art, the above detailed disclosure is merely an example and does not constitute a limitation on the present disclosure. Although not explicitly stated herein, various modifications, improvements, and amendments to the present disclosure may be made by a person skilled in the art. Such modifications, improvements, and amendments are suggested in the present disclosure. Therefore, such modifications, improvements, and amendments still fall within the spirit and scope of the exemplary embodiments of the present disclosure.

Finally, it should be understood that the embodiments described in the present disclosure are only used to illustrate principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A system for emergency supervision of overflow in smart city pipe networks based on an Internet of Things (IoT) large model, comprising an emergency supervision and management platform, wherein the emergency supervision and management platform is configured to:
obtain pipe network monitoring data via an emergency supervision object platform based on sensors deployed in a drainage pipe network;
generate overflow estimation data via a prediction model based on the pipe network monitoring data and future weather data;
in response to determining that the overflow estimation data satisfies an overflow condition, generate overflow control parameters based on the overflow estimation data, wherein the overflow control parameters include at least one of interception well parameters, pump station parameters, and regulation-and-storage facility parameters, and the pump station parameters include an operating power and a pumping direction of a target pump; and
send the overflow control parameters to the emergency supervision object platform and control operation parameters of an emergency overflow device, including:
controlling a gate opening degree of a sluice gate based on the interception well parameters, wherein the sluice gate is deployed at an overflow outlet of an interception well;
controlling the target pump to operate at the pumping direction and the operating power based on the pump station parameters;
controlling opening of a target pipeline valve to discharge water pumped by the target pump to a drainage region; and
controlling an inlet valve opening degree and an outlet valve opening degree of a regulation and storage facility based on the regulation-and-storage facility parameters;
wherein the regulation and storage facility is connected to the drainage pipe network.

2. The system of claim 1, wherein the prediction model includes a hydrological simulation model and a graph-based hydrological model, the hydrological simulation model is a physical model, and the graph-based hydrological model is a neural network model;
wherein the emergency supervision and management platform is further configured to:
determine a prediction horizon;
determine a prediction model type based on the prediction horizon;
in response to determining that the prediction model type is the graph-based hydrological model, construct an city pipe network graph based on structural data of the drainage pipe network, the pipe network monitoring data, and the future weather data; and
generate the overflow estimation data using the graph-based hydrological model based on the city pipe network graph.

3. The system of claim 2, wherein the emergency supervision and management platform is further configured to:
determine an overflow prototype pattern based on the future weather data;
determine a type of device to be controlled based on the overflow prototype pattern; and
determine the prediction horizon based on the type of device to be controlled.

4. The system of claim 2, wherein the emergency supervision and management platform is further configured to:
determine pipe network health data based on the pipe network monitoring data; and
construct the city pipe network graph based on the pipe network health data, the structural data of the drainage pipe network, and the pipe network monitoring data, and the future weather data.

5. The system of claim 4, wherein the emergency supervision and management platform is further configured to:
in response to determining that the pipe network health data indicates that a drainage pipeline satisfies a sediment cleaning condition, determine a flushing time based on the future weather data; and
generate a flushing control instruction and send the flushing control instruction to a flushing valve associated with the drainage pipeline at the flushing time to execute a pipeline flushing operation.

6. The system of claim 2, wherein the emergency supervision and management platform is further configured to:
obtain a future weather feature based on the future weather data;
determine a pipe network status feature based on the pipe network monitoring data and the structural data of the drainage pipe network;
determine model invocation parameters based on the future weather feature, the pipe network status feature, and the prediction horizon; wherein the model invocation parameters include an invocation time and the prediction model type corresponding to the invocation time; and
determine the prediction model type based on the model invocation parameters.

7. The system of claim 6, wherein the emergency supervision and management platform is further configured to:
in response to determining that a count of the prediction model type is greater than one, combine the overflow estimation data predicted by the prediction model according to the invocation time to obtain updated overflow estimation data.

8. The system of claim 1, wherein the emergency supervision and management platform is further configured to:
determine candidate control parameters based on the overflow estimation data; and
execute an iteration process based on the candidate control parameters and the prediction model, the iteration process including:
inputting the candidate control parameters of a current iteration into the prediction model to generate a comprehensive risk cost corresponding to the candidate control parameters of the current iteration; wherein the comprehensive risk cost is determined based on a predicted overflow volume of an overflow outlet output by the prediction model, an environmental risk weight, and an equipment operating cost;
in response to determining that the iteration process does not satisfy a stop condition, obtaining updated candidate control parameters based on the candidate control parameters of the current iteration and the comprehensive risk cost, designating the updated candidate control parameters as input for a next iteration; and
in response to determining that the iteration process satisfies the stop condition, stopping the iteration process and determining the overflow control parameters based on the candidate control parameters of the current iteration.

9. The system of claim 8, wherein the sensors further include a water quality sensor, and the water quality sensor is configured to obtain water quality sensor data;
wherein the emergency supervision and management platform is further configured to:
determine a pollutant concentration of the overflow outlet based on the water quality sensor data and the predicted overflow volume through a water quality prediction algorithm; and
determine the comprehensive risk cost based on the pollutant concentration.

10. The system of claim 9, wherein the emergency supervision and management platform is further configured to:
determine the pollutant concentration based on the water quality sensor data and pipe network health data through the water quality prediction algorithm.

11. A method for emergency supervision of overflow in smart-city pipe networks, wherein the method is executed by the emergency supervision and management platform of claim 1, the method comprising:
obtaining the pipe network monitoring data via the emergency supervision object platform based on the sensors deployed in the drainage pipe network;
generating the overflow estimation data via the prediction model based on the pipe network monitoring data and the future weather data;
in response to determining that the overflow estimation data satisfies the overflow condition, generating the overflow control parameters based on the overflow estimation data, wherein the overflow control parameters include at least one of the interception well parameters, the pump station parameters, and the regulation-and-storage facility parameters, and the pump station parameters include the operating power and the pumping direction of the target pump; and
sending the overflow control parameters to the emergency supervision object platform and controlling the operation parameters of the emergency overflow device, including:
controlling the gate opening degree of the sluice gate based on the interception well parameters, wherein the sluice gate is deployed at the overflow outlet of the interception well;
controlling the target pump to operate at the pumping direction and the operating power based on the pump station parameters;
controlling opening of the target pipeline valve to discharge water pumped by the target pump to the drainage region; and
controlling the inlet valve opening degree and the outlet valve opening degree of the regulation and storage facility based on the regulation-and-storage facility parameters;
wherein the regulation and storage facility is connected to the drainage pipe network.

12. The method of claim 11, wherein the prediction model includes a hydrological simulation model and a graph-based hydrological model, the hydrological simulation model is a physical model, and the graph-based hydrological model is a neural network model;
the generating the overflow estimation data via the prediction model based on the pipe network monitoring data and the future weather data includes:
determining a prediction horizon;
determining a prediction model type based on the prediction horizon;
in response to determining that the prediction model type is the graph-based hydrological model, constructing an city pipe network graph based on the structural data of the drainage pipe network, the pipe network monitoring data, and the future weather data; and
generating the overflow estimation data using the graph-based hydrological model based on the city pipe network graph.

13. The method of claim 12, wherein the determining a prediction horizon includes:
determining an overflow prototype pattern based on the future weather data;
determining a type of device to be controlled based on the overflow prototype pattern; and
determining the prediction horizon based on the type of device to be controlled.

14. The method of claim 12, wherein the constructing an city pipe network graph further includes:
determining pipe network health data based on the pipe network monitoring data; and
constructing the city pipe network graph based on the pipe network health data, the structural data of the drainage pipe network, the pipe network monitoring data, and the future weather data.

15. The method of claim 14, wherein the method further comprises:
in response to determining that the pipe network health data indicates that a drainage pipeline satisfies a sediment cleaning condition, determining a flushing time based on the future weather data; and
generating a flushing control instruction and sending the flushing control instruction to a flushing valve associated with the drainage pipeline at the flushing time to execute a pipeline flushing operation.

16. The method of claim 12, wherein the determining a prediction model type based on the prediction horizon includes:
obtaining a future weather feature based on the future weather data;
determining a pipe network status feature based on the pipe network monitoring data and the structural data of the drainage pipe network;
determining model invocation parameters based on the future weather feature, the pipe network status feature, and the prediction horizon; wherein the model invocation parameters include an invocation time and the prediction model type corresponding to the invocation time; and
determining the prediction model type based on the model invocation parameters.

17. The method of claim 16, wherein the method further comprises:
in response to determining that a count of the prediction model type is greater than one, combining the overflow estimation data predicted by the prediction model according to the invocation time to obtain updated overflow estimation data.

18. The method of claim 11, wherein the generating overflow control parameters based on the overflow estimation data includes:
determining candidate control parameters based on the overflow estimation data; and
executing an iteration process based on the candidate control parameters and the prediction model, the iteration process including:
inputting the candidate control parameters of a current iteration into the prediction model to generate a comprehensive risk cost corresponding to the candidate control parameters of the current iteration; wherein the comprehensive risk cost is determined based on a predicted overflow volume of an overflow outlet output by the prediction model, an environmental risk weight, and an equipment operating cost;
in response to determining that the iteration process does not satisfy a stop condition, obtaining updated candidate control parameters based on the candidate control parameters of the current iteration and the comprehensive risk cost, as the candidate control parameters for a next iteration; and
in response to determining that the iteration process satisfies the stop condition, stopping the iteration and determining the overflow control parameters based on the candidate control parameters of the current iteration.

19. The method of claim 18, wherein the sensors further include a water quality sensor, the water quality sensor being configured to obtain water quality sensor data;
the method further comprises:
determining a pollutant concentration of the overflow outlet based on the water quality sensor data and the predicted overflow volume through a water quality prediction algorithm; and
determining the comprehensive risk cost based on the pollutant concentration.

20. The method of claim 19, wherein the determining a pollutant concentration of the overflow outlet based on the water quality sensor data and the predicted overflow volume through a water quality prediction algorithm includes:
determining the pollutant concentration based on the water quality sensor data and pipe network health data through the water quality prediction algorithm.
